(19) United States
(12) Patent Application Publication
(10) Pub. No.: US 2026/0287566 A1
(43) Pub. Date: Sep. 24, 2026

(54) METHOD FOR QUANTITATIVE ANALYSIS OF LOADS INSIDE AND OUTSIDE CARBON NANOTUBE

(71) Applicant: Jiangsu University, Jiangsu (CN)

(72) Inventors: Bingliang ZHOU, Jiangsu (CN); Xiujuan LIU, Jiangsu (CN); Anthony DICHIARA, Jiangsu (CN); Nanqing LIU, Jiangsu (CN); Mei WANG, Jiangsu (CN)

(73) Assignee: Jiangsu University, Jiangsu (CN)

(21) Appl. No.: 18/868,782

(22) PCT Filed: Jan. 16, 2024
(86) PCT No.: PCT/CN2024/072493

§ 371 Date: Nov. 25, 2024

(30) Foreign Application Priority Data
Jul. 25, 2023 (CN) 202310921791.0

(51) Int. Cl. G01N 33/00; G01N 27/626; G01N 31/16

(52) U.S. Cl. CPC G01N 33/0003; G01N 27/626; G01N 31/16

(57) ABSTRACT

The present disclosure provides a method for quantitative analysis of loads inside and outside a carbon nanotube, including the following steps: (1) placing a carbon nanotube loaded with metal catalyst particles in a tube furnace, and vacuuming the tube furnace; (2) annealing and end-capping; (3) calcining to obtain a calcined carbon nanotube, adding the calcined carbon nanotube to a reactor, adding dilute sulfuric acid to the reactor, and heating and stirring to obtain a metal sulfate solution, and an end-capped carbon nanotube having metal catalyst particles loaded inside; (4) determining a nickel load inside the carbon nanotube by ICP-MS; and (5) determining a content of metal ions in the metal sulfate solution by EDTA complexometric titration.

### TECHNICAL FIELD

The present disclosure relates to the field of detection methods for element contents, and specifically to a method for quantitative analysis of loads inside and outside a carbon nanotube.

### BACKGROUND

Carbon nanotubes can serve as excellent catalyst supports due to their unique structures and properties. Catalysts can be assembled inside a carbon nanotube in an appropriate way, such that the catalytic activity of the catalyst can be improved due to an adjustment effect of a lumen of the carbon nanotube for performance of the catalyst. In most of the existing technologies, the catalyst is assembled on inner and outer walls of a carbon nanotube through liquid-phase impregnation. However, the uncertainty of a total amount of a precursor solution entering the carbon nanotube makes it impossible to control a final load of nickel nanoparticles in the carbon nanotube. Currently, the total metal load on the carbon nanotube can be detected by inductively coupled plasma mass spectrometry (ICP-MS) or flame atomic absorption spectroscopy. However, these detection methods do not have selectivity and cannot distinguish among absolute loads on inner and outer walls of a carbon nanotube. The carbon nanotube can be photographed locally by a transmission electron microscope to observe nickel loads on inner and outer walls of the carbon nanotube. However, this method has serious errors.

### SUMMARY

In view of the problem that the existing methods for detecting loads of carbon nanotubes do not have selectivity for metal nanoparticles loaded on inner and outer walls of a carbon nanotube and exhibit a low detection accuracy, the present disclosure provides a method for quantitative analysis of loads inside and outside a carbon nanotube. In the present disclosure, before detection, a carbon nanotube is end-capped in advance to prevent the leakage of metal nanoparticles loaded inside the carbon nanotube, which provides a prerequisite for the subsequent detection of loads on inner and outer walls of the carbon nanotube separately and improves a detection accuracy for loads inside and outside the carbon nanotube. Moreover, in the present disclosure, a carbon nanotube is end-capped during annealing, and a rate and a degree of an end-capping reaction are adjusted by adjusting an annealing temperature and an annealing rate to allow a prominent end-capping effect.

The present disclosure allows the above technical objective through the following technical means:

A method for quantitative analysis of loads inside and outside a carbon nanotube is provided, including the following steps:
S1, pretreatment: placing a carbon nanotube loaded with metal catalyst particles in a tube furnace, and vacuuming the tube furnace, where the metal catalyst particles are one selected from the group consisting of cobalt, nickel, and copper;
S2, end-capping: annealing the carbon nanotube at an annealing temperature of 1,200° C. to 1,500° C. to obtain an annealed carbon nanotube, where at beginning of cooling, a carbon source gas is introduced into the tube furnace;
S3, oxidation and hydrothermal reaction: calcining the annealed carbon nanotube obtained in the S2 in atmosphere such that the metal catalyst particles loaded on an outer wall of the annealed carbon nanotube are converted into an oxidation state to obtain a calcined carbon nanotube; placing the calcined carbon nanotube in a reactor, adding dilute sulfuric acid to the reactor, and heating and stirring such that oxidized metal catalyst particles attached to the outer wall of the carbon nanotube are dissolved to obtain a metal sulfate solution, and an end-capped carbon nanotube having the metal catalyst particles loaded inside;
S4, detection of a load inside the carbon nanotube: determining a load inside the end-capped carbon nanotube obtained in the S3 by IPC-MS; and
S5, detection of a load outside the carbon nanotube: determining a content of the metal catalyst particles in the metal sulfate solution obtained in the S3 by ethylenediaminetetraacetic acid (EDTA) complexometric titration, to obtain the load outside the carbon nanotube.

Further, in the S1, the tube furnace is vacuumed for 1 h to 2 h such that a vacuum degree in the tube furnace is 0.1 MPa, and after the vacuuming is completed, the tube furnace is sealed for 0.5 h to 1 h.

Further, in the S2, a heating rate in the tube furnace is controlled at 3° C./min to 5° C./min, and the annealing temperature is held for 10 h to 15 h.

Further, in the S2, the cooling is conducted as follows: cooling at a rate of 3° C./min to 5° C./min to 600° C., and then furnace-cooling to room temperature.

Further, in the S2, the carbon source gas is one or more selected from the group consisting of acetylene, methane, and ethylene.

Further, in the S2, the carbon source gas is introduced at a rate of 10 mL/min to 20 mL/min for 2 min to 5 min.

Further, in the S3, a concentration of the dilute sulfuric acid is 3.5 mol/L.

Further, in the S3, the stirring is conducted at 80° C. to 100° C. for 6 h or more; and the hydrothermal reaction is repeated 2 to 3 times.

Further, in the S4, specific operation steps of the IPC-MS are as follows:
S4.1, extracting the end-capped carbon nanotube having the metal catalyst particles loaded inside, adding an acid, heating for dissolution, and diluting to a predetermined volume to obtain a solution to be tested; and
S4.2, testing the solution to be tested from the S4.1 on an inductively coupled plasma mass spectrometer to obtain a content of the metal catalyst particles.

Further, in the S5, specific operation steps of the EDTA complexometric titration are as follows:
S5.1, preparing a pH buffer solution;
S5.2, adding the pH buffer solution dropwise to the metal sulfate solution to obtain a first mixed solution with a pH of 8 to 10;
S5.3, continuously adding an indicator dropwise to the first mixed solution until the first mixed solution is discolored to obtain a second mixed solution; and
S5.4, continuously adding an EDTA standard solution dropwise to the second mixed solution until the second mixed solution is discolored,
where a molar concentration of the EDTA standard solution is 0.2 mol/L, and a nickel content in a nickel sulfate aqueous solution is calculated according to a formula I:

$${W}_{Ni} = \frac{{c}_{EDTA} \times {v}_{EDTA} \times M}{m \times 1000} \times 1000 \qquad ( I )$$

where C_{EDTA} represents the molar concentration of the EDTA standard solution; V_{EDTA} represents a consumed volume of the EDTA standard solution; M represents a relative mass fraction of the metal catalyst particles; and m represents a mass of the carbon nanotube initially loaded with the metal catalyst particles.

The present disclosure has the following advantages:
1. The end-capping of a carbon nanotube in the present disclosure can prevent nickel nanoparticles inside the carbon nanotube from leaking, thereby improving a detection accuracy for a nickel load inside the carbon nanotube. The end-capping enables the separate detection of nickel loads inside and outside a carbon nanotube, thereby allowing the comprehensive evaluation of catalytic performance.
2. In the present disclosure, the end-capping is conducted at an annealing stage. At the annealing stage, a structure of a carbon nanotube undergoes rearrangement and repair to some extent, which is conducive to the filling of carbon atoms produced during decomposition of acetylene into an open end. Moreover, a rate and a degree of an end-capping reaction can be adjusted by controlling an annealing temperature and an annealing rate to allow a prominent end-capping effect for a carbon nanotube.

### BRIEF DESCRIPTION OF THE DRAWINGS

FIG. 1 shows X-ray diffraction (XRD) patterns of end-capped carbon nanotubes after aerobic calcination in Example 1 of the present disclosure;

FIG. 2(a) shows a transmission electron microscopy (TEM) image of a carbon nanotube before end-capping in Example 1 of the present disclosure;

FIG. 2(b) shows a TEM image of a carbon nanotube after end-capping in Example 1 of the present disclosure;

FIG. 3 is a comparison diagram of Raman characterization and ID/IG values of end-uncapped and end-capped carbon nanotubes in Example 1 of the present disclosure at different annealing temperatures; and

FIG. 4 is a comparison diagram of specific surface areas of and nickel loads inside end-uncapped and end-capped carbon nanotubes in Example 1 of the present disclosure at different annealing temperatures.

### DETAILED DESCRIPTION OF THE EMBODIMENTS

The present disclosure will be further described below in conjunction with the accompanying drawings and specific embodiments, but the protection scope of the present disclosure is not limited thereto.

### Example 1

In this example, metal catalyst particles were nickel, and a method for quantitative analysis of nickel loads inside and outside a carbon nanotube included the following steps:
S1: Pretreatment: A nickel-loaded carbon nanotube was placed in a tube furnace, and the tube furnace was vacuumed for 1 h until a vacuum degree in the tube furnace was 0.1 MPa to expel combustible gases.

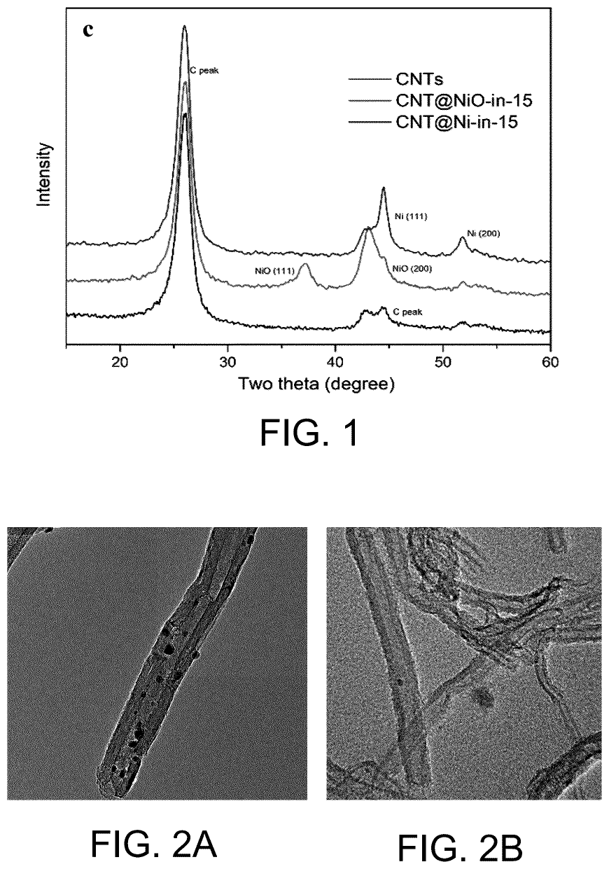

In this example, after the 1 h of vacuuming, the tube furnace was further sealed for 0.5 h, and the gas-tightness of the tube furnace was checked to ensure the smooth proceed of the subsequent process steps.
S2: End-capping: The nickel-loaded carbon nanotube was heated at a heating rate of 5° C./min to 1,450° C. and kept at 1,450° C. for 12 h, then cooled at a rate of 3° C./min to 600° C., and then furnace-cooled to room temperature. During the cooling, acetylene was introduced into the tube furnace for 2 min at a rate of 10 mL/min with the help of a flow meter to complete the end-capping for the carbon nanotube.
S3: Oxidation and hydrothermal reaction: An annealed carbon nanotube obtained in the S2 was calcined in the atmosphere for 3 h to 4 h such that elemental nickel loaded on an outer wall of the carbon nanotube was converted into an oxidation state. FIG. 1 shows XRD patterns of end-capped carbon nanotubes after aerobic calcination. It can be seen from this figure that a carbon nanotube (end-capped) after aerobic calcination includes a carbon nanotube support, elemental nickel loaded on an inner wall of the carbon nanotube support, and nickel oxide loaded on an outer wall of the carbon nanotube support. A calcined carbon nanotube was placed in a reactor, 30 mL of dilute sulfuric acid with a concentration of 3.5 mol/L was added to the reactor, and stirring was conducted at 90° C. for 6 h to obtain a yellow-green nickel sulfate aqueous solution, and an end-capped carbon nanotube having nickel loaded inside. The hydrothermal reaction was repeated 2 to 3 times to ensure that the elemental nickel on the outer wall of the carbon nanotube was completely converted into nickel sulfate.
S4: A nickel load inside the carbon nanotube was determined by IPC-MS through the following specific steps:
S4.1: The end-capped carbon nanotube having nickel loaded inside was extracted, an acid was added, heating was conducted for dissolution, and dilution was conducted to a predetermined volume to obtain a solution to be tested.
S4.2: The solution to be tested from the S4.1 was tested on an inductively coupled plasma mass spectrometer to obtain a nickel content.

S5: A nickel content in the nickel sulfate aqueous solution was determined by EDTA complexometric titration through the following specific steps:

S5.1: Ammonium hydroxide and ammonium chloride were weighed and prepared into a buffer solution with a pH of 10.

S5.2: The buffer solution was added dropwise to the nickel sulfate aqueous solution to obtain a first mixed solution with a pH of 10.

S5.3: An indicator murexide was continuously added dropwise to the first mixed solution until the first mixed solution turned golden-yellow to obtain a second mixed solution.

S5.4: An EDTA standard solution was continuously added dropwise to the second mixed solution until the second mixed solution turned violet-blue.

A molar concentration of the EDTA standard solution was 0.2 mol/L. The nickel content in the nickel sulfate aqueous solution was calculated according to a formula I:

$${W}_{Ni} = \frac{{c}_{EDTA} \times {v}_{EDTA} \times M}{m \times 1000} \times 1000 \qquad ( I )$$

where C_{EDTA} represents the molar concentration of the EDTA standard solution; V_{EDTA} represents a consumed volume of the EDTA standard solution; M_{Ni} represents a relative mass fraction of nickel; and m represents a mass of the initial nickel-loaded carbon nanotube.

In order to verify the influence of end-capping on a detection accuracy for nickel loads on inner and outer walls of a carbon nanotube, the following control experiment was further conducted in this example:
S1: An end-uncapped carbon nanotube was calcined in the atmosphere for 3 h to 4 h such that elemental nickel loaded on the carbon nanotube was converted into nickel oxide to obtain a calcined carbon nanotube. The calcined carbon nanotube was placed in a reactor, 30 mL of dilute sulfuric acid with a concentration of 3.5 mol/L was added to the reactor, and stirring was conducted at 90° C. for 6 h to obtain a yellow-green nickel sulfate aqueous solution and an end-uncapped carbon nanotube. The hydrothermal reaction was repeated 2 to 3 times to ensure that nickel oxide on the carbon nanotube was completely converted into nickel sulfate.
S2: A nickel content in the nickel sulfate aqueous solution was determined by EDTA complexometric titration through the following specific steps:
S2.1: Ammonium hydroxide and ammonium chloride were weighed and prepared into a buffer solution with a pH of 10.
S2.2: The buffer solution was added dropwise to the nickel sulfate aqueous solution to obtain a first mixed solution with a pH of 10.
S2.3: An indicator murexide was continuously added dropwise to the first mixed solution until the first mixed solution turned golden-yellow to obtain a second mixed solution.
S2.4: An EDTA standard solution was continuously added dropwise to the second mixed solution until the second mixed solution turned violet-blue.

A molar concentration of the EDTA standard solution was 0.2 mol/L. The nickel content in the nickel sulfate aqueous solution was calculated according to a formula I:

$${W}_{Ni} = \frac{{c}_{EDTA} \times {v}_{EDTA} \times M}{m \times 1000} \times 1000 \qquad ( I )$$

where C_{EDTA} represents the molar concentration of the EDTA standard solution; V_{EDTA} represents a consumed volume of the EDTA standard solution; M_{Ni} represents a relative mass fraction of nickel; and m represents a mass of the initial nickel-loaded carbon nanotube.

FIG. 2(a) and FIG. 2(b) show SEM images of the end-uncapped and end-capped carbon nanotubes in this example, respectively. It can be seen from this figure that a black passivation layer is formed at ends of the end-capped carbon nanotube, indicating that a prominent end-capping effect is allowed with acetylene during annealing in this example.

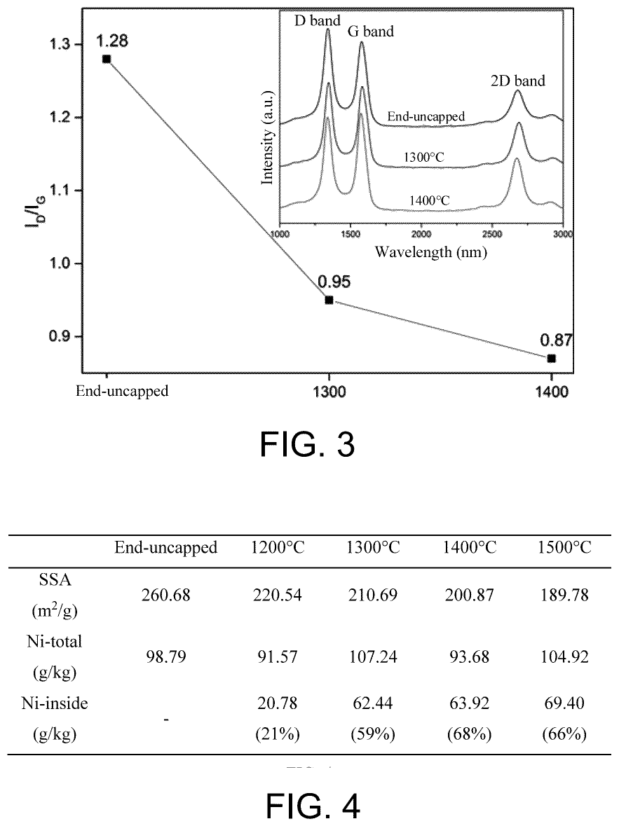

FIG. 3 is a comparison diagram of Raman characterization and ID/IG values of the end-uncapped and end-capped carbon nanotubes in this example at different annealing temperatures. It can be seen from this figure that an ID/IG value of the end-uncapped carbon nanotube is significantly higher than an ID/IG value of the end-capped carbon nanotube, indicating that the end-uncapped carbon nanotube has relatively-low crystallinity and many defects.

FIG. 4 is a comparison diagram of specific surface areas of and nickel loads inside the end-uncapped and end-capped carbon nanotubes in this example at different annealing temperatures. It can be seen from this figure that nickel loads inside and outside the end-uncapped carbon nanotube cannot be detected separately, but only a total nickel load on the end-uncapped carbon nanotube can be detected. However, a ratio of nickel loads inside and outside the end-capped carbon nanotube increases with the increase of an annealing temperature, and there is an optimal end-capping effect for the carbon nanotube at an annealing temperature of about 1,400° C.

### Example 2

This example was different from Example 1 in that: metal catalyst particles were cobalt, and a method for quantitative analysis of cobalt loads inside and outside a carbon nanotube was the same as the method for quantitative analysis of nickel loads inside and outside a carbon nanotube in Example 1 merely except that the pH buffer solution and the indicator in the S5 had different compositions.

### Example 3

This example was different from Example 1 in that: metal catalyst particles were copper, and a method for quantitative analysis of copper loads inside and outside a carbon nanotube was the same as the method for quantitative analysis of nickel loads inside and outside a carbon nanotube in Example 1 merely except that the pH buffer solution and the indicator in the S5 had different compositions.

The above examples are preferred implementations of the present disclosure, but the present disclosure is not limited to the above implementations. Any obvious improvement, substitution, or modification made by those skilled in the art without departing from the essence of the present disclosure should fall within the protection scope of the present disclosure.

To the claims:

1. A method for quantitative analysis of loads inside and outside a carbon nanotube, comprising the following steps:
S1, pretreatment: placing a carbon nanotube loaded with metal catalyst particles in a tube furnace, and vacuuming, wherein the metal catalyst particles are one selected from the group consisting of cobalt, nickel, and copper;
S2, end-capping: annealing the carbon nanotube, wherein an annealing temperature is set to 1,200° C. to 1,500° C.; and at beginning of cooling, a carbon source gas is introduced into the tube furnace;
S3, oxidation and hydrothermal reaction: calcining the annealed carbon nanotube obtained in the S2 in atmosphere such that the metal catalyst particles loaded on an outer wall of the carbon nanotube are converted into an oxidation state; then placing in a reactor, adding dilute sulfuric acid to the reactor, and heating and stirring such that oxidized metal catalyst particles attached to the outer wall of the carbon nanotube are dissolved to obtain a metal sulfate solution, and an end-capped carbon nanotube having the metal catalyst particles loaded inside;
S4, detection of a load inside the carbon nanotube: determining a load inside the end-capped carbon nanotube obtained in the S3 by inductively coupled plasma mass spectrometry; and
S5, detection of a load outside the carbon nanotube: determining a content of the metal catalyst particles in the metal sulfate solution obtained in the S3 by ethylenediaminetetraacetic acid complexometric titration, to obtain the load outside the carbon nanotube.

2. The method for quantitative analysis according to claim 1, wherein in the S1, vacuum is processed for 1 h to 2 h such that a vacuum degree in the tube furnace is 0.1 MPa, and after the vacuuming is completed, the tube furnace is sealed for 0.5 h to 1 h.

3. The method for quantitative analysis according to claim 1, wherein in the S2, a heating rate in the tube furnace is controlled at 3° C./min to 5° C./min, and the annealing temperature is held for 10 h to 15 h.

4. The method for quantitative analysis according to claim 1, wherein in the S2, the cooling is conducted as follows: cooling at a rate of 3° C./min to 5° C./min to 600° C., and then furnace-cooling to room temperature.

5. The method for quantitative analysis according to claim 1, wherein in the S2, the carbon source gas is one or more selected from the group consisting of acetylene, methane, and ethylene.

6. The method for quantitative analysis according to claim 1, wherein in the S2, the carbon source gas is introduced at a rate of 10 mL/min to 20 mL/min for 2 min to 5 min.

7. The method for quantitative analysis according to claim 1, wherein in the S3, a concentration of the dilute sulfuric acid is 3.5 mol/L.

8. The method for quantitative analysis according to claim 1, wherein in the S3, the stirring is conducted at 80° C. to 100° C. for 6 h or more;
and the hydrothermal reaction is repeated 2 to 3 times.

9. The method for quantitative analysis according to claim 1, wherein in the S4, specific operation steps of the inductively coupled plasma mass spectrometry are as follows:
S4.1, extracting the end-capped carbon nanotube having the metal catalyst particles loaded inside, adding an acid, heating for dissolution, and diluting to a predetermined volume to obtain a solution to be tested; and
S4.2, testing the solution to be tested from the S4.1 on an inductively coupled plasma mass spectrometer to obtain a content of the metal catalyst particles.

10. The method for quantitative analysis according to claim 1, wherein in the S5, specific operation steps of the ethylenediaminetetraacetic acid complexometric titration are as follows:
S5.1, preparing a pH buffer solution;
S5.2, adding the pH buffer solution dropwise to the metal sulfate solution to obtain a first mixed solution with a pH of 8 to 10;
S5.3, continuously adding an indicator dropwise to the first mixed solution until the first mixed solution is discolored to obtain a second mixed solution; and
S5.4, continuously adding an ethylenediaminetetraacetic acid standard solution dropwise to the second mixed solution until the second mixed solution is discolored,
wherein a molar concentration of the ethylenediaminetetraacetic acid standard solution is 0.2 mol/L, and a nickel content in a nickel sulfate aqueous solution is calculated according to a formula I:
$${W}_{Ni} = \frac{{c}_{EDTA} \times {v}_{EDTA} \times M}{m \times 1000} \times 1000 \qquad ( I )$$
wherein C_{EDTA} represents the molar concentration of the ethylenediaminetetraacetic acid standard solution; V_{EDTA} represents a consumed volume of the ethylenediaminetetraacetic acid standard solution; M represents a relative mass fraction of the metal catalyst particles; and m represents a mass of the carbon nanotube initially loaded with the metal catalyst particles.
